(19) United States
(12) Patent Application Publication
(10) Pub. No.: US 2022/0313615 A1
(43) Pub. Date: Oct. 6, 2022

(54) Nanoparticle Capable of Loading and Releasing Active Constituents, Production Method and Application Thereof

(71) Applicant: Feng Chia University, Taichung City (TW)

(72) Inventor: Chun-Feng Lai, Taichung City (TW)

(21) Appl. No.: 17/586,978

(22) Filed: Jan. 28, 2022

(30) Foreign Application Priority Data
Apr. 1, 2021 (TW) 110112256

(51) Int. Cl. A61K 9/51; A61K 9/00; A61K 47/02

(52) U.S. Cl. CPC A61K 9/5115; A61K 9/0051; A61K 47/02; B82Y 5/00

(57) ABSTRACT

A nanoparticle capable of loading and releasing active constituents includes many mesopores with a large pore size and can be produced by one-step synthesis. The nanoparticle has a BET surface area over 100 m²/g and an average pore size over 1 nm that can load and release a large amount of active constituents quickly to improve the performance and applicability of related slow release carriers. In addition to its application in the field of drug-loaded contact lenses, the properties of such nanoparticle can be applied for releasing other active constituents or drugs, and used for the treatment of human body or even for the release of environmental active constituents.

### FIELD OF THE INVENTION

The present invention relates to nanoparticles, and more particularly to a nanoparticle having mesopores with high BET surface area and large pore size and capable of loading and releasing active constituents, and its production method and related applications.

In an application of the nanoparticle capable of loading and releasing active constituents in accordance with the present invention, the nanoparticle is used for loading an ocular drug or moisturizing active constituent and combining the drug to a contact lens as a functional contact lens, and this application will be described in details below. It is noteworthy that the nanoparticle provided by the present invention is not limited to a single application only, any similar, equivalent, or derived scope of applicability is intended to be covered within the scope of the appended claims of the present invention.

### BACKGROUND OF THE INVENTION

At present, the basic method to treat eye diseases is mainly to apply eye drops to a patient's eyes. However, human eyes have a self-protection mechanism, so that after users apply the eye drops to their eyes, their eye blink reflex makes the eye drops to flow out, or the application of eye drops may stimulate tears. As a result, the eye drops will flow out with the tears altogether, and the medicinal effect will be naturally compromised.

In addition, drug active constituents in the eye drops with a short ocular surface retention time will pass through the tear ducts and enter into the nasal cavity. Due to poor corneal permeability, metabolic degradation, or other factors, the bioavailability of the eye drops is low under the traditional mode of medication, and it is necessary to apply the eye drops frequently, thereby leading to poor comfort and low patient compliance. In the meantime, the concentration of the drug in the eyes fluctuates, and thus the systemic absorption of the drug will cause toxic and adverse reaction in the eyes or the whole body. In order to improve the comfort of wearing contact lenses, moisturizing the lenses is very important to the eyes. Most of the contact lenses on the market are put into a preservation solution added with a moisturizing constituent to achieve the moisturizing effect. However, the moisturizing effect of this method does not last long.

In view of the aforementioned problems, it is a subject for related manufacturers to lock the moisturizing active constituent into the contact lens and limit the moisturizing active constituent to release slowly over time instead of losing quickly, so as to maintain the comfort of the eyes for a long time. Obviously, finding a quick and effective way of releasing the active constituent without affecting the comfort of the eyes to overcome the problems of the prior art demands immediate attentions and feasible solutions.

### SUMMARY OF THE INVENTION

Therefore, it is a primary objective of the present invention to provide a nanoparticle capable of loading and releasing active constituents and its production method and application overcome to overcome the problems of the prior art including the limitation of administrating the eye drops for the treatment of eye diseases and the discomfort to the user eyes. The present invention discloses a nanoparticle capable of loading and releasing active constituents, and its production method and related applications to alleviate or at least provide a concrete and feasible alternative to solve the existing technical problems. The first inventive concept of the present invention is to provide a nanoparticle capable of loading and releasing active constituents, which has a plurality of mesopores with a large pore size, a BET surface area over 100 m²/g and an average pore size over 1 nm.

The second inventive concept of the present invention is to provide a nanoparticle production method comprising the steps of: preparing and mixing hexadecyltrimethylammonium p-toluenesulphonate, trolamine, and pure water to produce a mixture, and heating the mixture to 50 degrees Centigrade for one hour; increasing and maintaining the temperature at 60 degrees Centigrade, while adding tetraethyl orthosilicate to the mixture for the synthesis of a product; and purifying the product before sintering the product at 550 degrees Centigrade for 6 hours to obtain the nanoparticle.

The third inventive concept of the present invention is to apply the nanoparticle produced by the aforementioned production method to an ophthalmic device, and the nanoparticles capable of loading and releasing active constituents are distributed on the ophthalmic device containing hydrogel or silicone hydrogel.

According to the above description, compared with the traditional mode of administration of an eye drop, the drug-loaded contact lens can greatly improve the ocular surface retention time of the drug, significantly increase the bioavailability of the eye drop from 1%˜5% to 72.5%˜100.0%, and reduce the systemic absorption of the drug in the eyes and the toxic and adverse reaction in the whole body, and thus the invention provides the most ideal mode of administration.

The nanoparticle of the present invention has high BET surface area, large pore size, and the capability of quickly loading a large quantity of active constituent and slowly releasing the active constituent to improve the performance and applicability of related slow release carriers. In addition to the application applied to the field of drug-loaded contact lenses as illustrated by the following embodiments, the nanoparticle of the invention can also be applied for releasing other active constituents or drugs for the treatment of human body, or even for absorbing harmful substances in the environment.

### BRIEF DESCRIPTION OF THE DRAWINGS

Preferred embodiments are used together with the attached drawings for the detailed description of the invention. It is intended that the embodiments and figures disclosed herein are to be considered illustrative rather than restrictive.

FIGS. 1A˜1D are transmission electron microscope (TEM) images of the nanoparticles capable of loading and releasing active constituents in accordance with several preferred embodiments of the present invention respectively;

FIG. 2A is a flow chart of a nanoparticle production method in accordance with the present invention;

FIG. 2B shows the modification process of a nanoparticle having a complex functional group in accordance with the present invention;

FIGS. 3A and 3B are a schematic view and a cross-sectional view of an ophthalmic device in accordance with a first preferred embodiment of the present invention respectively;

FIGS. 3C-3E are schematic views of two other ophthalmic devices of the present invention;

FIG. 4 is a schematic view showing the process of producing an ophthalmic device in accordance with a second preferred embodiment of the present invention;

FIG. 5 is a schematic view showing the process of producing an ophthalmic device in accordance with a third preferred embodiment of the present invention;

FIG. 6 is a spectrogram showing the modification of a nanoparticle functional group having a complex functional group in accordance with the present invention;

FIG. 7A is a cross-sectional view of a contact lens manufactured by the ophthalmic device production method in accordance with the second and third embodiments of the present invention;

FIG. 7B is an optical zone transmittance diagram of the contact lens manufactured by the aforementioned ophthalmic device production method in accordance with an embodiment of the present invention;

FIGS. 8A and 8B are an absorbance rate test diagram and a release rate test diagram of the drug test 1 of the present invention drug test 1 respectively;

FIG. 9 is a release rate test diagram of the drug test 2.1 of the present invention;

FIG. 10 is a release rate test diagram of the drug test 2.2 of the present invention;

FIG. 11 is a release rate test diagram of the drug test 2.3 of the present invention;

FIG. 12 is a release rate test diagram of the drug test 3 of the present invention;

FIG. 13 is a release rate test diagram of the drug test 4 of the present invention;

FIG. 14 is a release rate test diagram of the drug test 5 of the present invention; and

FIG. 15 is an absorbance and wavelength test diagram of the glycosaminoglycan test of the present invention.

### DESCRIPTION OF THE PREFERRED EMBODIMENTS

To make it easier for our examiner and people having ordinary skill in the art to understand the technical characteristics of the invention we use preferred embodiments together with the attached drawings for the detailed description of the invention, in which like reference numerals refer to like parts or operations. It is noteworthy that the embodiments and drawings are used for the purpose of describing and illustrating the technical characteristics of the invention, but not intended to limit the scope of the invention.

In the description of the disclosure of the present invention, the terms “a”, “one”, “a kind of”, and/or “the” are used as a unit, an element and a component for the description of this specification to facilitate the description and provide a general meaning to the scope of the invention, so that both “a” and “one” refer to one or at least one including an odd or even number, unless otherwise specified.

In this invention, the terms “comprising”, “including”, “having” “containing” or any other similar terminologies intend to cover non-exclusive contents. For example, the invention comprising an element of a plurality of elements, structures, products, or devices is not limited to the elements listed in the specification only, but also including other usually inherent elements, structures, products or devices which are not listed specifically. Unless otherwise specified, the term “or” refers to the inclusive “or”, but not the exclusive “or”.

In the present invention, a procedure of a flow chart is used to describe the manufacturing and operation steps carried out by a system in accordance with an embodiment of the present invention. It should be understood that the steps are not necessarily carried out according to a specific order. On the contrary, the steps may be carried out in a reverse order or at the same time. In addition, other operations may be added to these processes, or one or more steps may be omitted to achieve similar or same effects.

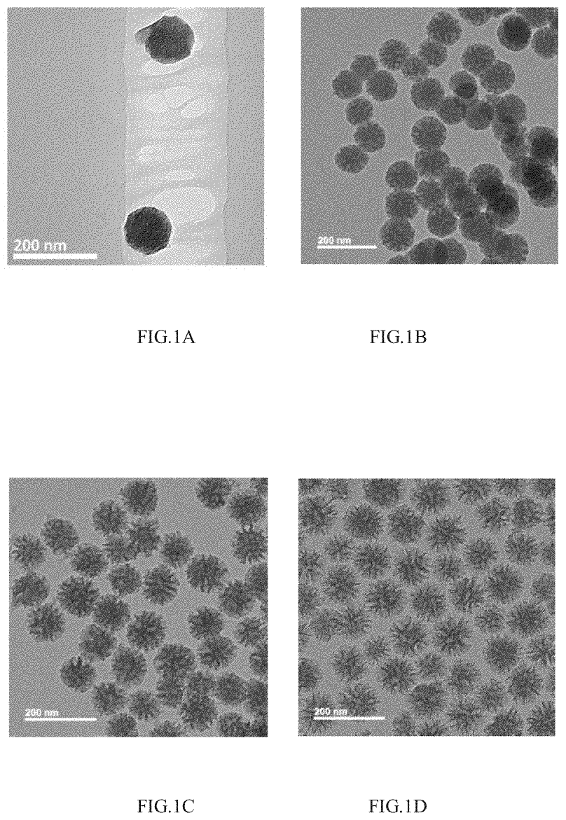

With reference to FIGS. 1 for a nanoparticle capable of loading and releasing active constituents in accordance with the present invention, FIGS. 1A, 1B, 1C, and 1D show the first to fourth embodiments of the present invention respectively, and the nanoparticle has a plurality of mesopores with different appearances and pore sizes, a BET surface area over 100 m²/g, an average pore size over 1 nm, a pore volume over 0.10 cm³/g to 5.0 cm³/g and a particle diameter falling within a range of 10˜500 nm. With reference to FIGS. 1A—1D for the nanoparticles having different BET surface areas in accordance to different embodiments of the present invention respectively, the BET surface area and mesopore size as shown in FIGS. 1A, 1B, 1C and 1D increase incrementally. On the other hand, the structure of the nanoparticle of the present invention may include a plurality of sheet overlapping structures extending in all directions from the center of the particle to the outside, and a plurality of mesopores with different pore sizes distributed on the sheet overlapping structures, or the structure of the nanoparticle of the present invention may include a plurality of dendritic structures extending in all directions from the center to the outside, and a plurality of mesopores with different pore sizes distributed on the dendritic structures.

Wherein, the average pore size is preferably 2 nm, and may be below 50 nm; the BET surface area of serval preferred embodiments of the present invention is preferably over 300 m²/g, more preferably over 600 m²/g, and further more preferably over 800 m²/g; the pore size is preferably over 3 nm, more preferably over 10 nm, and further more preferably 20 nm, and the preferred pore size is from 3 nm for the small pore size to 50 nm for the large pore size; the pore volume is preferably over 0.5 cm³/g to 5.0 cm³/g, more preferably over 1.0 cm³/g to 5.0 cm³/g, and further more preferably over 1.5 cm³/g to 5.0 cm³/g; and the particle diameter is preferably 60˜150 nm.

The nanoparticle is preferably made of silicon dioxide (also known as silica) having large-pore mesoporous silica nanoparticles (LPMSNs), which is the so-called the sheet overlapping structure or the dendritic structure shown in FIGS. 1A˜1D.

TABLE 1
BET surface area, pore volume and pore size of the nanoparticle
in accordance with the embodiments shown in FIGS. 1A~1D

| | BET Surface Area | Pore Volume | Pore Size |
|---|---|---|---|
| Embodiment | (m²/g) | (cm³/g) | (nm) |
| Nanoparticle 1 | 158.27 | 0.169 | 3.9 |
| Nanoparticle 2 | 587.15 | 0.47 | 7.46 |
| Nanoparticle 3 | 486.10 | 0.87 | 13.8 |
| Nanoparticle 4 | 647.48 | 1.45 | 20.0 |

Production Method of Nanoparticle

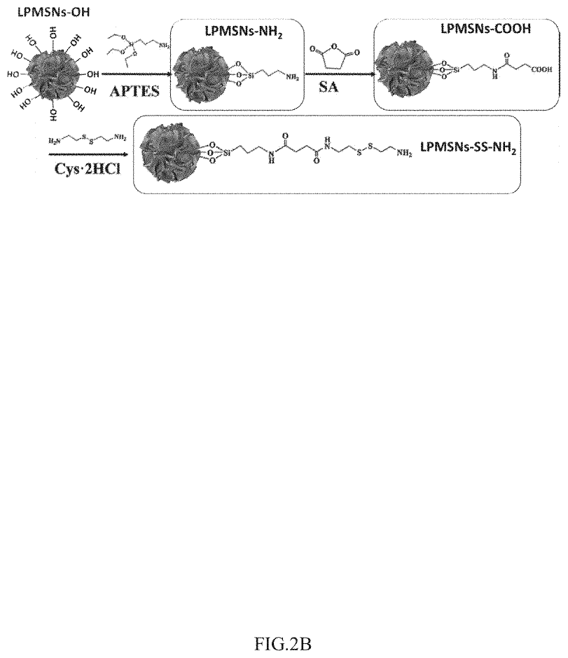

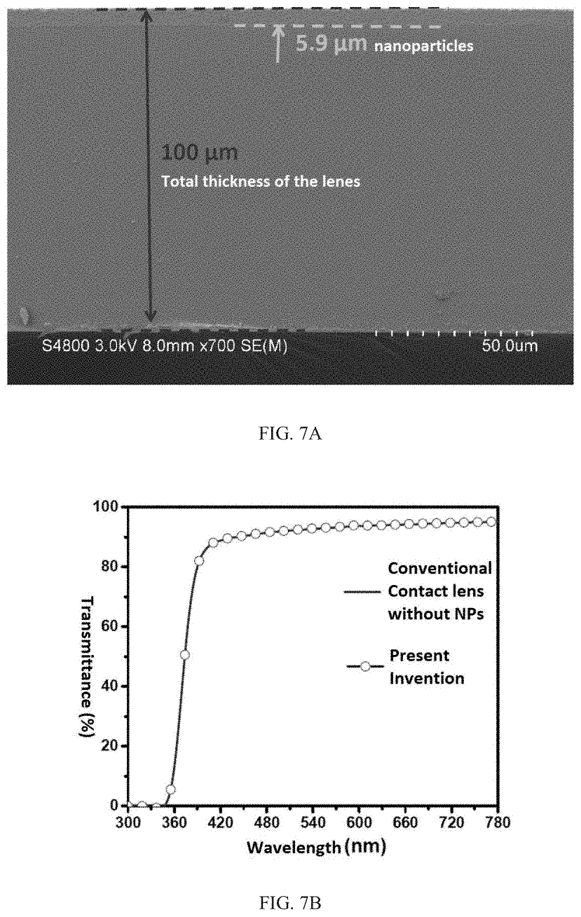

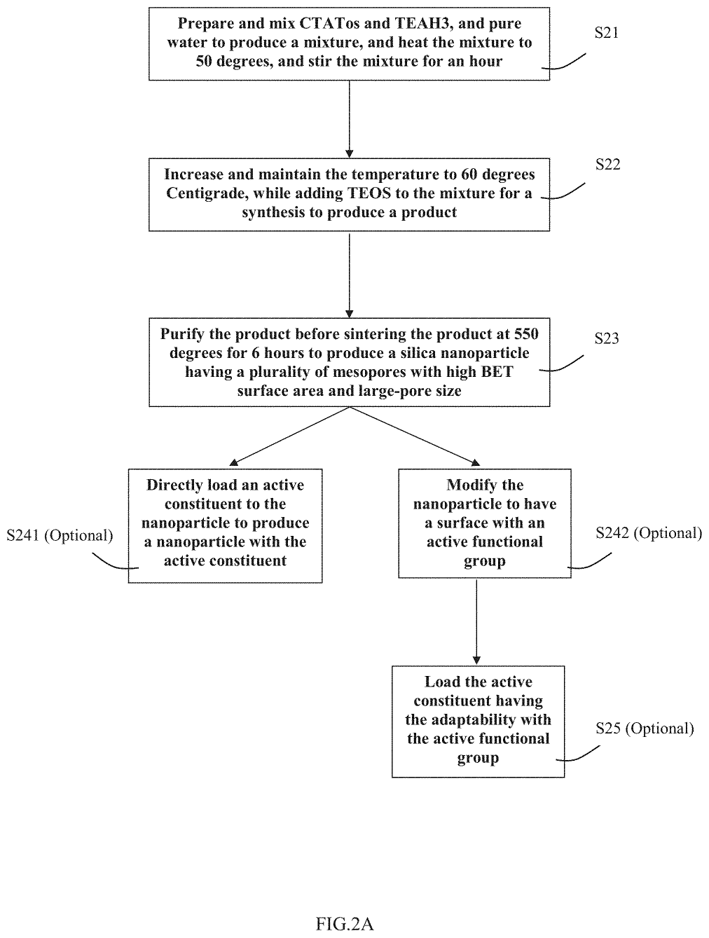

With reference to FIG. 2A for the nanoparticle production method, the method includes the following steps. Step S21: Prepare and mix hexadecyltrimethylammonium p-toluenesulphonate (CTATos), trolamine (TEAH3), and pure water to produce a mixture, and heat the mixture to 50 degrees Centigrade, and stir the mixture for an hour;

Step S22: Increase and maintain the temperature to 60 degrees Centigrade, while adding tetraethyl orthosilicate (TEOS) to the mixture for a synthesis to produce a product; and

Step S23: Purify the product before sintering the product at 550 degrees Centigrade for 6 hours to produce a silica nanoparticle having a plurality of mesopores with high BET surface area and large-pore size, and the nanoparticle with the mesopores can be directly introduced to the manufacturing process of an ophthalmic device such as contact lens.

Wherein, the technical characteristic of the present invention is shown in the Steps S21 and S22, a lower temperature of 50 degrees Centigrade is used to stir and dissolve the related compositions, and then the temperature is increased to 60 degrees Centigrade for the synthesis in order to form a nanoparticle structure having mesopores of different pore sizes. IN the Step S22, the concentration of TEOS will affect the size of the synthesized nanoparticle, wherein the nanoparticle particle of the present invention has a diameter falling within a range of 10˜500nm. In the meantime, the nanoparticle produced by the one-step synthesis of the present invention has a pore size from 3 nm for the small pore size to 50 nm for the large pore size and an ability of loading the active constituents of different molecular weights.

For the nanoparticle of the present invention or the nanoparticle produced by the production method of the present invention, the following steps may be added optionally to a manufacturing process of an ophthalmic device, wherein the nanoparticle production method can further include the following steps:

Step S241: Directly load an active constituent to the nanoparticle to produce a nanoparticle with the active constituent; or Step S242 (optional): Modify the nanoparticle to have a surface with an active functional group, and then Step S25 (optional): Load the active constituent having the adaptability with the active functional group, and the active constituent refers to a biological cell of tissue having active constituents with a molecular weight preferably from 2 to 400,000 g/mole and including drug, gas, vitamin, glycosaminoglycan or biomacromolecule, wherein the eye medication provided by the present invention includes but not limited to an antihistamine drug (Ketotifen fumarate salt), a drug for the treatment of myopia (Atropine, Atropine sulfate salt monohydrate), a dry eye syndrome drug (lifitegrast), a broad-spectrum antibiotic (Chlorhexidine), an anti-inflammatory and analgesic drug (Diclofenac), an eye drop uses as an antibiotic (Levofloxacin), a glaucoma drug (Timolol maleate salt, Dorzolamide, Pilocarpine), a local anesthetic (Lidocaine, Bupivacaine, Tetracaine) or an artificial synthetic corticosteroid (Dexamethasone), a drug for allergic conjunctivitis (Sodium cromoglicate), etc. or even a combination of several drugs to achieve a compound effect or acts as a compound reagent to inhibit side effects. The gas may be hydrogen or carbon dioxide, etc. The vitamin may include vitamin B2, vitamin B6, vitamin E, vitamin B12, etc. The glycosaminoglycan may be hyaluronic acid or trehalose, etc. The biomacromolecule may be collagen, etc. In the steps of the present invention, the nanoparticle surface is formed with an active functional group that can be bonded with the active constituent, so that the active constituent is loaded in the nanoparticle to achieve the effect of stably attach the active constituent, and the active constituent can be stably attached to the active constituent in a general non-release environment.

With reference to the following Table 2 for the examples of the present invention applicable for the load activity specification and their corresponding molecular weights, but this table only lists the exemplary applicable constituents, but the active constituent of the present invention is not limited to those listed in this table.

| | | Molecular |
|---|---|---|
| | | Weight |
| Name | CAS No. | (g/mole) |
| Hydrogen (H2) | — | 2 |
| Carbon dioxide (CO2) | — | 44 |
| Ketotifen fumarate salt (antihistamine) | 34580-14-8 | 425.5 |
| Timolol maleate salt (glaucoma) | 26921-17-5 | 432.5 |
| Atropine (myopia) | 51-55-8 | 289.37 |
| Atropine sulfate salt monohydrate | 5908-99-6 | 694.83 |
| (myopia) | | |
| Lifitegrast (dry eye syndrome) | 102596-78-5 | 615.48 |
| Fluorometholone (conjunctivitis) | 426-13-1 | 376.47 |
| Ciprofloxacin (bacterial corneal ulcer, | 85721-33-1 | 331.34 |
| bacterial conjunctivitis) | | |
| Cyclosporine | 59865-13-3 | 1202.61 |
| Lidocaine | 137-58-6 | 234.34 |
| Local anesthetic | | |
| Loteprednol etabonate | 82034-46-6 | 466.9 |
| (Corticosteroids for the treatment of eye | | |
| inflammation) | | |
| Natamycin | 7681-93-8 | 665.733 |
| (Antifungal drug for the treatment of | | |
| fungal infection around the eye) | | |
| Prednisolone | 50-24-8 | 360.44 |
| (for the treatment of arthritis) | | |
| Vitamin B2 (Riboflavin) | 83-88-5 | 376.36 |
| Vitamin B12 | 68-19-9 | 1355.37 |
| Vitamin B6 (Pyridoxine) | 65-23-6 | 169.18 |
| Vitamin E | 59-02-9 | 430.71 |
| Lutein | 127-40-2 | 568.87 |
| Taurine | 107-35-7 | 125.15 |
| Hyaluronic acid | 9004-61-9 | 3000 to 400000 |
| Trehalose | 6138-23-4 | 378.33 |

The active functional group of the modified nanoparticle as described in the Step S242 of the present invention preferably includes a hydroxyl group (—OH), a carboxylic acid group (—COOH), an amine group (—NH₂), an acrylic group, a sulfhydryl group (—SH), a disulfide bond (S—S) or a compound functional group formed by combining a plurality function groups, etc. depending on the subsequent loaded active constituent of the nanoparticle. In a preferred embodiment as shown in FIG. 2B, a solvent (alcohol) is added to the product obtained in the Step S23 in a single-neck bottle for the dispersion by ultrasonic shock, and then (3-aminipropyl) triethoxysilane (APTES) is added and stirred at room temperature, and then purified by centrifugation, and then further purified by the solvent (alcohol) to obtain the modified the nanoparticle with an amine group (—NH₂) formed on its surface. In another preferred embodiment, the nanoparticle can be formed into a compound functional group with the amine group and the disulfide bond (S—S) sequentially by the constituents including 3-aminopropyltriethoxysilane (APTES), succinic anhydride (SA), and cystamine dihydrochloride (cys2HCl).

Further, the method of the present invention further includes the Step S25 of loading the active constituent, and a mixing method or a soaking method is mainly used to load an active constituent such as an antihistamine drug (Ketotifen fumarate salt), wherein the antihistamine drug is mixed into a table salt solution, and the nanoparticle (or the nanoparticle with its carrier such a contact lens or any other suitable carrier is soaked into the antihistamine drug aqueous solution to absorb the drug for over 8 hours, so as to obtain the nanoparticle (or its carrier) loaded with the active constituent (which is the antihistamine drug in this example).

The nanoparticle (or its carrier loaded with the active constituent is preferably put in a simulated human tear solution environment (with a pH value about 7.0 to 7.5) during the process of releasing the active constituent, so that the active constituent will start releasing in a slow manner.

Application in Ophthalmic Device

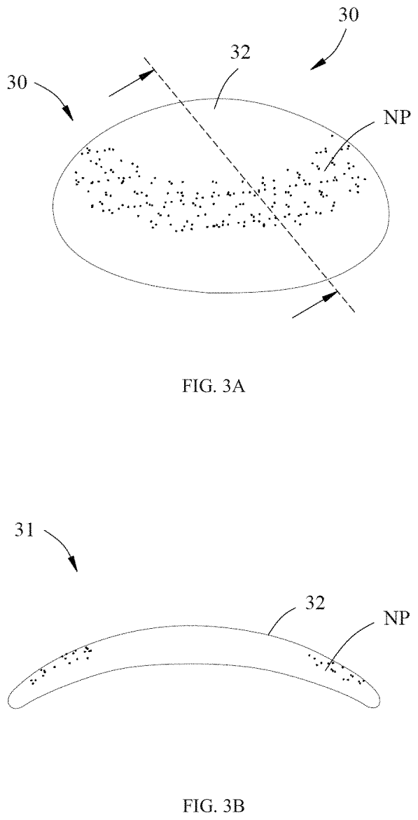

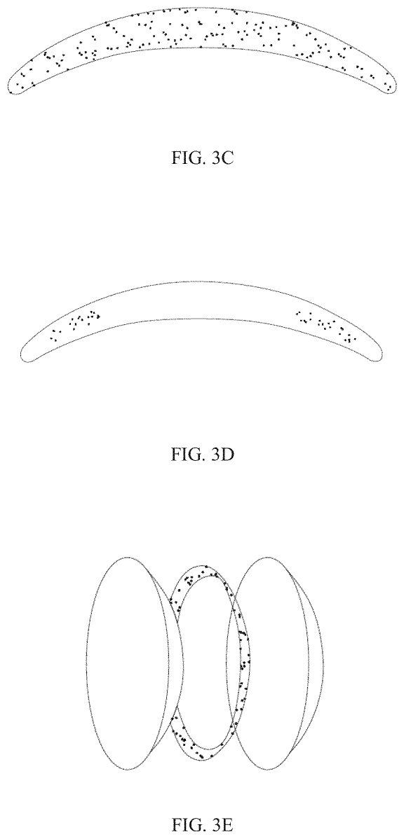

The nanoparticle of the present invention can be combined with an artificial intraocular lens, a contact lens material, an ophthalmic film (or an ocular inserts) to produce an ophthalmic device 30. With reference to FIGS. 3A and 3B for the schematic view and the cross-sectional view of an ophthalmic device in accordance with the first preferred embodiment of the present invention respectively, a contact lens 31 is used as the ophthalmic device 30 in this embodiment, and the contact lens 31 of the first preferred embodiment is one having the nanoparticle (labeled as NP in FIG. 3) circularly distributed around an optical zone 32 of the contact lens material, and the optical zone 32 is the reachable range of the arc of the contact lens, which is the lens area with the refractive power, but the cross section as shown in FIG. 3B does not show that the nanoparticle NP is particularly protruded from the material of the contact lens 31, and the material of the contact lens 31 is aligned flatly to provide a smooth surface. Since the nanoparticle loaded with the active constituent in accordance with the invention has high BET surface area and large pore size, the nanoparticle can load a large amount of active constituents and release drug with high efficiency.

With reference to FIG. 3C for a contact lens 31 in accordance with the second preferred embodiment of the invention, the nanoparticles are distributed uniformly on the material of the whole contact lens 31. With reference to FIG. 3E for a contact lens 31 in accordance with the third preferred embodiment of the invention, a sandwich structure similar to the one as shown in FIG. 3E is formed inside the contact lens 31, and the nanoparticles NP as shown in FIG. 3E are also formed into a circular shape around the periphery of the optical zone 32, and the top and bottom are clamped by the contact lens material to form the sandwich structure.

To produce the ophthalmic device of the aforementioned contact lens, the present invention provides at least three different production methods. In the method as shown in FIG. 3C, the nanoparticles are combined with the contact lens, and the compositions of the contact lens include two main types; hydrogel and silicone hydrogel, wherein the formula of hydrogel includes hydrogel (HEMA), ethyleneglycoldimethacrylate (EGDMA), HMPP(2-hydroxy-2-methyl-l-phenyl-1-propanone), UV absorbent and an active constituent unmodified but loaded with the nanoparticles, which is formed into a mixed solution and then injected into a lower mold of a mold, and then pressed by a corresponding upper mold and cured by ultraviolet light The dry lens of the cured contact lens loaded with the nanoparticles is peeled off from the mold. After the dry lens is separated from the mold, the cured contact lens is put into water for hydration, and then a high-temperature sterilization process is performed, and then the loading of the active constituent is carried out as described in the Step S25. For example, the contact lens is preserved in a preservation solution containing the active constituent to allow the adsorption of the active constituent up to a saturated level.

Wherein, the hydrogel is formed by the polymerization of one or more one or more macromolecular monomers including (hydroxyethyl)methacrylate (HEMA), ethyleneglycoldimethacrylate (EGDMA), N-Vinylpyrrolidone, and poly(methyl methacrylate) (PMMA), and different (light or thermal) initiators contain azobisisobutyronitrile or 2,2′-Azobis(2-methylpropionitrile) (AIBN), (phenyl-azotriphenylmethane), tert-butyl-peroxide (TBP), cumyl peroxide, benzoyl peroxide (BPO), tert-butyl perbenzoate (TBPB) etc., and the optical initiator can be made of at least one of 2,4,6-trimethylbenzoyldiphenyl phosphine oxide (TPO), 2-Hydroxy-2-Methyl-l-phenyl-1-Porpanone (HMPP), 1-hydroxy cyclohexyl phenyl ketone, and (phenyl bis(2,4,6-trimethylbenzoyl)-phosphine oxide).

The silicone hydrogel includes at least one of (hydroxyethyl)methacrylate (HEMA), methyl methacrylate (MMA, acrylic monomer), polydimethylsiloxane (PDMS), PEG-PDMS methacrylate, N-vinylpyrrolidone, tetra(ethylene glycol) dimethacrylate, ethylene glycol methyl ether methacrylate or azobisisobutyronitrile or 2,2′-azobis(2-methylpropionitrile) (AIBN).

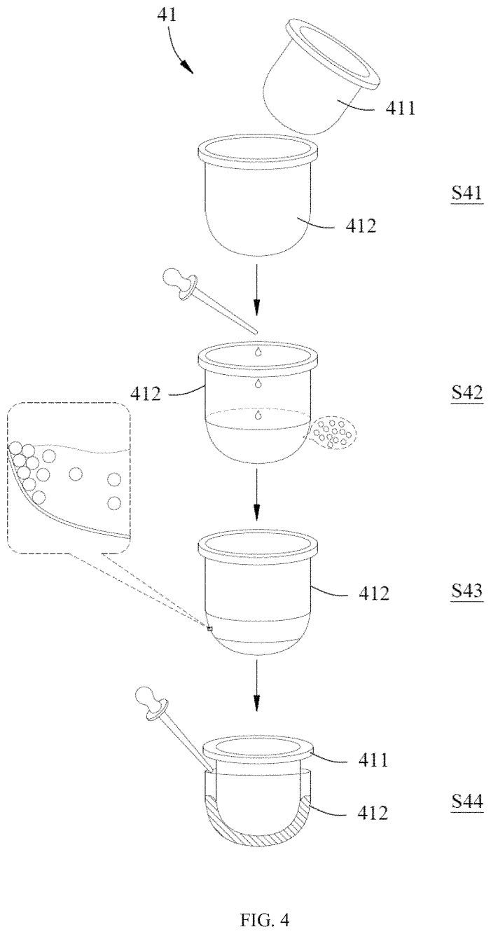

With reference to FIG. 4, corresponding to the preferred embodiment of FIGS. 3A and 3B, for the production method of the ophthalmic device of the contact lens in accordance with the second preferred embodiment of this invention, the method includes the following steps:

Step S41: Prepare a contact lens mold 41 including an upper mold 411 and a lower mold 412, wherein the bottom of the lower mold 412 of the contact lens mold 41 is an arc-shaped container structure adaptable to an eyeball arc, and the upper mold 411 is in a shape corresponding to the shape of the lower mold 412. However, when the upper mold 411 and the lower mold 412 are pressed and engaged with each other, a space is reserved for the material of the contact lens 31;

Step S42: Inject a solution formed by the nanoparticle loaded with the active constituent and the liquid-state material of the contact lens 31 into the lower mold 412, so that the bottom of the contact lens mold 41 is filled up with the nanoparticle solution;

Step S43: Evaporate the liquid-state material of the contact lens 31 and nanoparticle liquid solution loaded with active constituents in an evaporation process, wherein the nanoparticles will be precipitated in a circular distribution around the periphery of the optical zone according to the principle of thermodynamics;

Step S44: Inject the liquid-state material of the contact lens 31 containing the mixed solution of hydrogel or silicone hydrogel into the lower mold 412 and press the upper mold 411, and process subsequent operations such as curing and demolding and optional rough edge removal;

Step S45: (optional, not shown in the figure): After the hydration and packaging, a high-temperature and high-pressure sterilization is performed to obtain a conventional contact lens finished product.

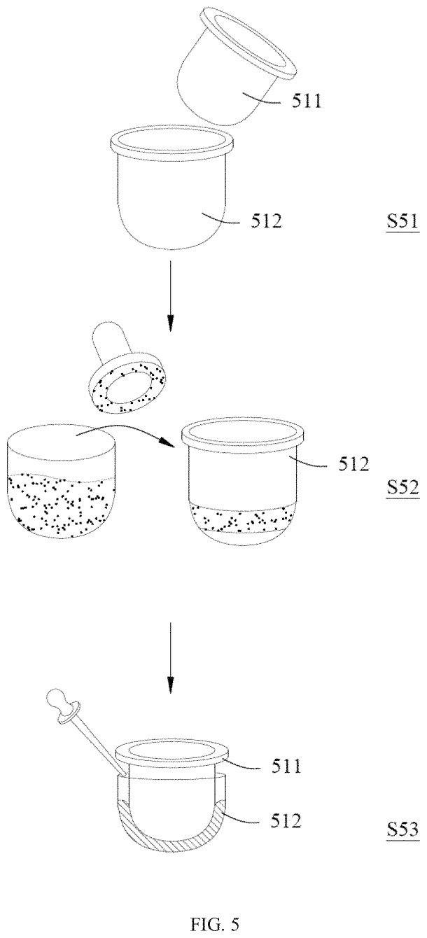

With reference to FIG. 5, corresponding to the preferred embodiment as shown in FIGS. 3A and 3B, for the production method of the ophthalmic device of the contact lens in accordance with the third preferred embodiment of this invention, the method includes the following steps:

Step S51: Prepare a contact lens mold 51, wherein the bottom of the contact lens mold 51 is an arc-shaped container structure adaptable to an eyeball arc. Similarly, the contact lens mold 51 includes an upper mold 511 and a lower mold 512, and the bottom of the lower mold 512 of the contact lens mold 51 is an arc-shaped container structure adaptable to an eyeball arc, and the upper mold 511 is in a shape corresponding to the shape of the lower mold 512. However, when the upper mold 511 and the lower mold 512 are pressed and engaged with each other, a space is reserved for the material of the contact lens 31;

Step S52: Prepare a ring-shaped embossing part 52, and dip the embossing part 52 into an active constituent solution (such as an alcohol, ketone, or ester solution containing an active constituent) containing nanoparticles loaded with the active constituent, and then attach the circular distributed nanoparticles loaded with the active constituent to the bottom of the lower mold 512 by embossing, and preform a curing (by a method including but not limited to light curing or thermal curing);

Step S53: Inject the liquid-state material of the contact lens 31 of the mixed hydrogel or silicone hydrogel solution into the lower mold 512, and press the upper mold 511, and process subsequent operations such as curing and demolding, and optionally removing rough edges;

Step S54: (optional, not shown in the figure): After the hydration and packaging, a high-temperature and high-pressure sterilization is performed to obtain a conventional contact lens finished product.

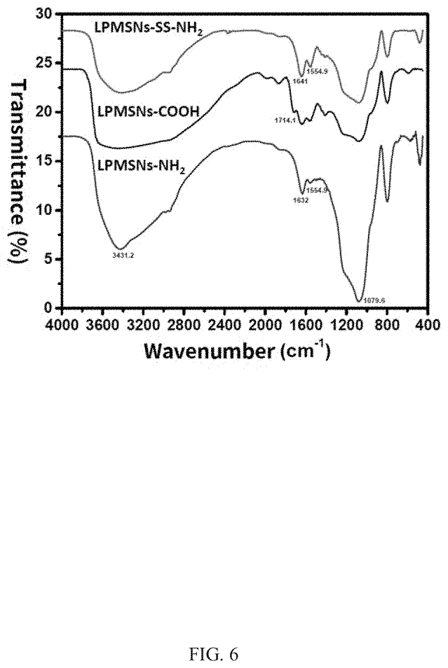

In the embodiment as shown in FIG. 3E, basically, the molding process or embossing process as shown in FIG. 4 can be used, and it is only necessary to prepare a layer of the contact lens 31 before configuring the nanoparticles, and then fabricate the nanoparticles in a ring shape, and finally follow the subsequent process as shown in FIGS. 5 and 6 to form the sandwich structure as shown in FIG. 3E.

The aforementioned Steps S45˜S44 and S55˜S53 are the commercial packaging steps of the contact lens, and the ophthalmic device production method of the present invention optionally includes these commercial packaging steps, but it is noteworthy that the nanoparticle of the present invention has a functional group with special structure and grafting adaptable to the functional group and can be well combined with the active constituent, so that the active constituent will not be lost during the autoclaving process, and the active constituent will be firmly attached to the nanoparticle.

However, the prescription or preparation method of the contact lens of the present invention is not limited to such manufacturing method only, but any conventional contact lens prescription or formation technology can be combined with this method for the combination of the nanoparticle, and the aforementioned contact lens of the present invention is only a carrier of the nanoparticle, and it is not intended to limit the nanoparticle that can only be combined with the contact lens. In fact, any suitable carrier should be covered within the scope of the present invention.

With reference to FIG. 7A for a cross-sectional view of a contact lens manufactured by the ophthalmic device production method in accordance with the second and third embodiments of the present invention, the nanoparticles loaded with the active constituent are uniformly and thinly distributed onto an area as shown in the upper half FIG. 7A, wherein the thickness is approximately 5.9 μm, and the total thickness of the material of the contact lens is approximately 100 μm.

Validation Experiment

The contact lens made of the nanoparticles loaded with the active constituent is used to carry out experiments to provide related descriptions and related data support of the loading and release performance.

With reference to FIG. 6 a spectrogram showing the modification of a nanoparticle functional group having a complex functional group in accordance with the present invention (for example, FIG. 2B with Transmittance (%)-wavenumber (cm⁻¹)), and the result indicates that the present invention sure can form the complex functional group having an amine group (—NH₂), a carboxylic acid group (—COOH) and a bisulfide bond (S—S).

In FIG. 7B and Table 3, an optical test of the contact lens in accordance with the embodiment as shown in FIG. 7A is performed, and the transmittance of an optical zone is tested by UV-B (280˜315 nm), UV-A (316˜380nm) and visible light (380˜780 nm), and the test results in Table 3 show that even though the contact lens of the present invention is attached with the nanoparticles, its optical performance will not be affected, and the contact lens of the invention is still the same as the conventional contact lens and complies with the international ANSI Z80.20 Class II specification.

TABLE 3

| Sample | UV-B | UV-A | Visible Light |
|---|---|---|---|
| Conventional contact | 0.0% | 14.5% | 92.2% |
| lens without loaded | | | |
| nanoparticles | | | |
| The present invention | 0.0% | 14.5% | 92.3% |

Drug Test 1—Ketotifen Fumarate Salt:

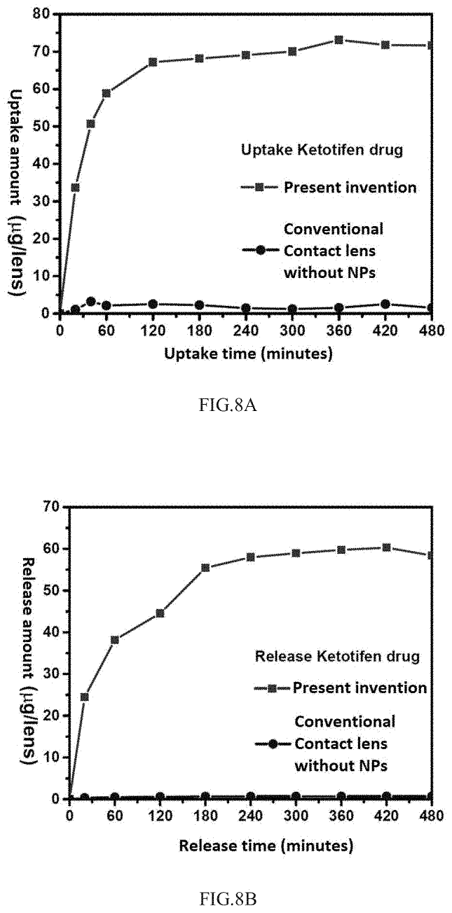

A hydrogel type contact lens containing 38% HEMA is prepared, and the hydrogel type contact lens manufactured by the process of this invention containing an active constituent (Ketotifen fumarate salt) loaded with nanoparticles (with a pore size of 7 nm) same as that containing the 38% HEMA are used for the absorption and release comparison test of the active constituents, and the nanoparticle loaded with an active constituent with a drug concentration of 5 m—wt % (50 μg/mL), and soaked and loaded by a fixed amount of 3 mL, and the nanoparticle layer formed on the contact lens has a thickness of approximately 6 μm. The two samples are processed with a high-temperature high-pressure sterilization before performing the drug release test. The test results are shown in FIG. 8A for the absorption test, and FIG. 8B for the release test as well as Table 4 below. The conventional contact lens without loaded nanoparticles has an absorption and release quantity of the active constituent much smaller than that of the present invention, which can show that of the nanoparticle provided by the present invention can improve the contact lens's ability of loading and releasing active constituents.

| | | Ketotifen fumarate salt | | |
|---|---|---|---|---|
| | | | | Drug |
| | | Uptake | Release | utilization rate |
| | Time | amount | amount | (Release/ |
| Name | (min.) | (μg/lens) | (μg/lens) | Absorption %) |
| Conventional contact | 480 | 3.23 | 0.70 | 21.7% |
| lens (made of a | | | | |
| silicone hydrogel | | | | |
| material) without | | | | |
| loaded nanoparticles | | | | |
| The present invention | 480 | 73.12 | 60.27 | 82.4% |
| (nanoparticle pore | | | | |
| size about 7 nm) | | | | |

Drug Test 2.1—Atropine Sulfate Salt Monohydrate (ASM):

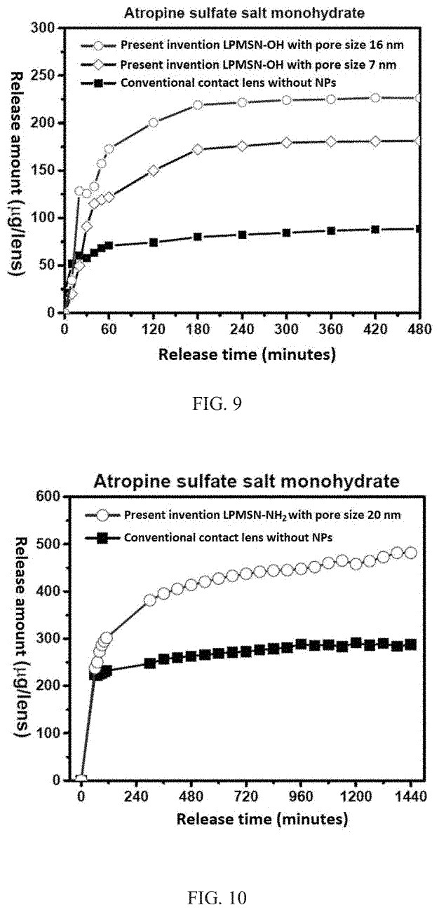

The ketotifen fumarate salt used for the drug test 1 is changed to an ASM drug, which is dipped in a load with a concentration of 0.1 wt % (1 mg/mL), but a nanoparticle layer with a thickness of approximately 10 nm is formed on the silicone hydrogel contact lens of this embodiment, and the nanoparticle used in this embodiment has been modified to have the —OH functional group, and the mesopore formed on the nanoparticle has a pore size of 7 nm or 16 nm. The result of the release amount is shown in FIG. 9 and Table 5 below.

TABLE 5

| | | ASM |
|---|---|---|
| | Time | Release Amount |
| Name | (min.) | (μg/lens) |
| Conventional contact lens | 480 | 88.53 |
| (made of a silicone | | |
| hydrogel material) without | | |
| loaded nanoparticles | | |
| The present invention | 480 | 181.28 |
| (nanoparticle —OH | | |
| pore size about 7 nm) | | |
| The present invention | 480 | 226.35 |
| (nanoparticle —OH | | |
| pore size about 16 nm) | | |

Drug Test 2.2—Atropine Sulfate Salt Monohydrate (ASM):

Similarly, the ASM drug is prepared and dipped in a load with a concentration of 0.5 wt %, and a nanoparticle layer with a thickness of approximately 10 nm is also formed on the hydrogel material contact lens in this embodiment, and the nanoparticle is formed on the contact lens without being loaded with an active constituent, but is directly dipped in the ASM drug solution for loading. The nanoparticle used in this embodiment has been modified to have the —NH₂ functional group, and the mesopores formed on the nanoparticle have a pore size of 20 nm. The result of the release test is shown in FIG. 10 and table 6 below.

TABLE 6

| | | ASM |
|---|---|---|
| | Time | Release Amount |
| Name | (min.) | (μg/lens) |
| Conventional contact lens | 1440 | 287.6 |
| (made of a silicone | | |
| hydrogel material) without | | |
| loaded nanoparticle | | |
| The present invention | 1440 | 481.4 |
| (nanoparticle —NH₂ | | |
| pore size about 20 nm) | | |

Drug Test 2.3—Atropine Sulfate Salt Monohydrate (ASM):

Similarly, the ASM drug is prepared and dipped in a load with a concentration of 0.5 wt %, and a nanoparticle layer with a thickness of approximately 10 m is also formed on the silicone hydrogel material contact lens in this embodiment, and the nanoparticle is formed on the contact lens without being loaded with an active constituent, but is directly dipped in the ASM drug solution for loading. The nanoparticle used in this embodiment has been modified to have the —NH₂ functional group, and the mesopores formed on the nanoparticle have a pore size of 20 nm. The result of the release test is shown in FIG. 11 and Table 7 below.

TABLE 7

| | | ASM |
|---|---|---|
| | Time | Release Amount |
| Name | (min.) | (μg/lens) |
| Conventional contact lens | 1440 | 504.2 |
| (made of a silicone | | |
| hydrogel material) without | | |
| loaded nanoparticles | | |
| The present invention | 1440 | 909.3 |
| (nanoparticle —NH₂ | | |
| pore size about 20 nm) | | |

Drug Test 3—Trehalose:

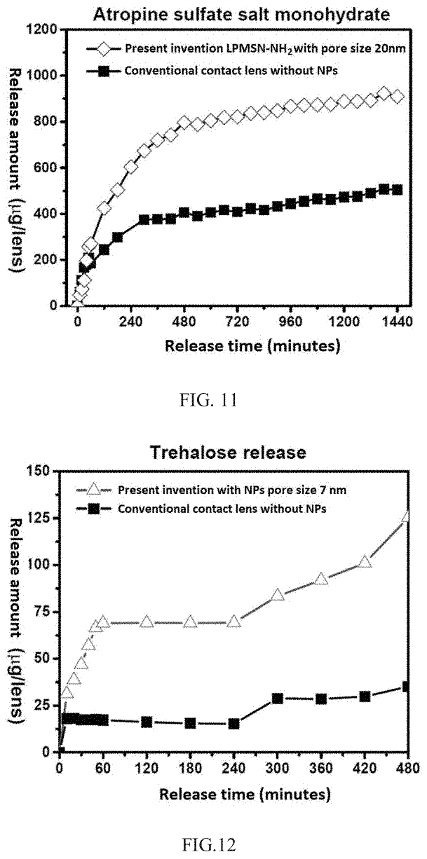

The sample of the drug test 1 is replaced by a Trehalose drug and dipped in an active constituent with a concentration of 50 m—wt % (500 μg/mL), and a nanoparticle layer with a thickness of approximately 10 μgm is formed on the silicone hydrogel material of the contact lens of this embodiment. The result of the release test is shown in FIG. 12 and Table 8 below.

TABLE 8

| | | Trehalose |
|---|---|---|
| | Time | Release Amount |
| Name | (min.) | (μg/lens) |
| Conventional contact lens | 480 | 35.12 |
| (made of a silicone | | |
| hydrogel material) without | | |
| loaded nanoparticles | | |
| The present invention | 480 | 125.32 |
| (nanoparticle pore size | | |
| about 7 nm) | | |

Drug Test 4—Vitamin B2:

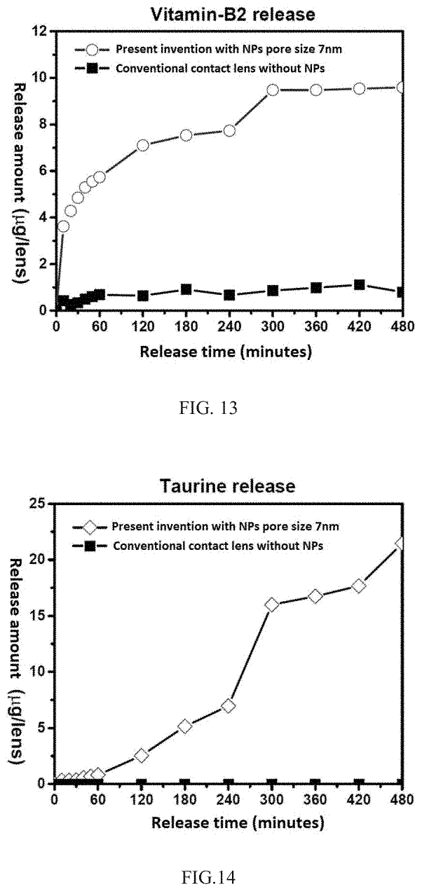

The sample of the drug test 1 is replaced by Vitamin B2, and dipped in an active constituent with a concentration of 1 m—wt % (5 μg/mL) and a nanoparticle layer with a thickness of approximately 10 μm is formed on the silicone hydrogel material of the contact lens of this embodiment. The result of the release test is shown in FIG. 13 and Table 9 below.

TABLE 9

| | | Vitamin B2 |
|---|---|---|
| | Time | Release Amount |
| Name | (min.) | (μg/lens) |
| Conventional contact lens | 480 | 0.8 |
| (made of a silicone | | |
| hydrogel material) without | | |
| loaded nanoparticles | | |
| The present invention | 480 | 9.59 |
| (nanoparticle pore size | | |
| about 7 nm) | | |

Drug Test 5—Taurine:

The sample of the drug test 1 is replaced by a Taurine drug, and dipped in an active constituent with a concentration of 5 m—wt % (50 μg/mL) and a nanoparticle layer with a thickness of approximately 10 μm is formed on the silicone hydrogel material of the contact lens of this embodiment. The result of the release test is shown in FIG. 14 and Table 10 below.

TABLE 10

| | | Taurine |
|---|---|---|
| | Time | Release Amount |
| Name | (min.) | (μg/lens) |
| Conventional contact lens | 480 | 0 |
| (made of a silicone | | |
| hydrogel material) without | | |
| loaded nanoparticles | | |
| The present invention | 480 | 21.46 |
| (nanoparticle pore size | | |
| about 7 nm) | | |

Biomacromolecular Test—Hyaluronic Acid HA3000

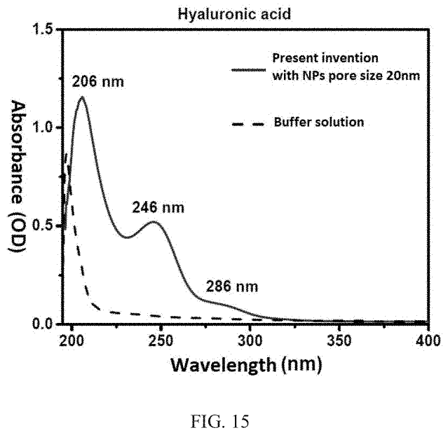

The sample of the drug test 1 is replaced by hyaluronic acid HA3000 and dipped in an active constituent with the same concentration. The result is shown in FIG. 15. In the nanoparticle of the present invention, the absorption peak with the adsorption wavelength of hyaluronic acid shows up at (206 nm, 246 nm, and 286 nm). Compared with the control solution (which is a buffer solution), the absorption peak with the adsorption wavelength of hyaluronic acid does not show up, thus proving that the nanoparticle of the present invention having a pore size of approximately 20 nm has the function of loading biomacromolecules of hyaluronic acid (with a molecular weight of 3000).

In the description of constituents and numbers for attribute quantities in some embodiments of the present invention, it should be understood that the terms “about”, “approximately” or “substantially” used for describing a certain quantity or number implies a tolerance of at least ±20%. In some embodiments, the numerical values or parameters used in the specification and claims are approximate values which may vary according to different features of individual embodiments. In some embodiments, the numerical value or parameter should take the required digits into account and adopt a general round up method. Even though a numerical value or parameter used in the certain embodiment to confirm the scope of the present invention is an approximate value, the setting of such numerical value should be as accurate as possible within the feasible range.

While the invention has been described by means of specific embodiments, numerous modifications and variations could be made thereto by those skilled in the art without departing from the scope and spirit of the invention as set forth in the claims.

What is claimed is:

1. A nanoparticle capable of loading and releasing active constituents, comprising a plurality of mesopores with a large pore size, a BET surface area over 100 m²/g and an average pore size over 1 nm.

2. The nanoparticle capable of loading and releasing active constituents according to claim 1, wherein the nanoparticle is made of silicon dioxide.

3. The nanoparticle capable of loading and releasing active constituents according to claim 1, wherein the nanoparticle surface comprises a hydroxyl group, a carboxylic acid group, an amine group, an acrylic group, a sulfhydryl group or an active functional group of any combination thereof, for loading an active constituent which is reactive to a biological cell or tissue, and the active constituent has a molecular weight between 2 to 400,000 g/mole.

4. The nanoparticle capable of loading and releasing active constituents according to claim 3, wherein the active constituent comprises a drug, a gas, a vitamin, a glycosaminoglycan or a biomacromolecule.

5. A production method of a nanoparticle capable of loading and releasing active constituents, comprising the steps of:
preparing and mixing hexadecyltrimethylammonium p-toluenesulphonate, trolamine and pure water to produce a mixture, and heating the mixture to 50 degrees Centigrade for an hour;
increasing and maintaining the temperature of the mixture at 60 degrees Centigrade, while adding tetraethyl orthosilicate to the mixture for a synthesis of a product; and
purifying the product before sintering the product at 550 degrees Centigrade for 6 hours to obtain the nanoparticle.

6. The production method of a nanoparticle capable of loading and releasing active constituents according to claim 5, wherein the nanoparticle is further processed with a surface modification, so that the surface of the nanoparticle has a hydroxyl group, a carboxylic acid group, an amine group, an acrylic group, a sulfhydryl group or an active functional group of any combination thereof.

7. The production method of a nanoparticle capable of loading and releasing active constituents according to claim 6, wherein the modified nanoparticle further loads an active constituent which is reactive to a biological cell or tissue, and the active constituent has a molecular weight between 2 to 400,000 g/mole.

8. An ophthalmic device, comprising the nanoparticle capable of loading and releasing active constituents according to claim 1, and the nanoparticle being distributed on the ophthalmic device including a hydrogel or a silicone hydrogel.

9. The ophthalmic device according to claim 8, wherein the nanoparticles are distributed at the periphery of an optical zone of the ophthalmic device to form a ring distribution or uniform distribution in the ophthalmic device.

10. The ophthalmic device according to claim 9, wherein the nanoparticles are disposed at the periphery of the optical zone of the ophthalmic device to form a ring distribution or clamped inside the ophthalmic device at the same time to define a sandwich structure.

11. The ophthalmic device according to claim 8, wherein the nanoparticle capable of loading and releasing active constituents is fixed and distributed on the ophthalmic device by a molding process or an embossing process.

12. The ophthalmic device according to claim 8, wherein when the ophthalmic device with the nanoparticle capable of loading and releasing active constituents is processed with a high-temperature high-pressure sterilization, the active constituent will not be released or fallen off from the nanoparticle.

13. The ophthalmic device according to claim 8, wherein the ophthalmic device comprises an artificial intraocular lens, a contact lens, or an ophthalmic film.

14. The ophthalmic device according to claim 8, wherein the hydrogel or the silicone hydrogel comprises hydroxyethyl methacrylate.
